(19) United States
(12) Patent Application Publication
(10) Pub. No.: US 2010/0056822 A1
(43) Pub. Date: Mar. 4, 2010

(54) METHOD FOR PRODUCING ISOCYANATES

(75) Inventors: Andreas Daiss, Deidesheim (DE); Andreas Woelfert, Bad Rappenau (DE); Carsten Knoesche, Niederkirchen (DE); Eckhard Stroefer, Mannheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/447,607

(22) PCT Filed: Nov. 6, 2007
(86) PCT No.: PCT/EP2007/061941

§ 371 Date: Apr. 28, 2009

(30) Foreign Application Priority Data
Nov. 7, 2006 (EP) 06123621.2

(51) Int. Cl. C07C 263/10

(52) U.S. Cl. 560/347

(57) ABSTRACT

The invention relates to a process for preparing isocyanates in the gas phase.

The present invention relates to a process for preparing isocyanates in the gas phase.

Polyisocyanates are produced in large quantities and serve mainly as starting materials for producing polyurethanes. They are usually prepared by reacting the corresponding amines with phosgene.

One possible way of preparing isocyanates is reaction in the gas phase. The advantages of this mode of operation are a reduced phosgene holdup, avoidance of intermediates which are difficult to phosgenate and increased reaction yields. Apart from effective mixing of the feedstreams, achievement of a narrow residence time spectrum and adherence to a narrow residence time window are important prerequisites for such a process to be able to be carried out industrially. These requirements can be met, for example, by the use of tube reactors operated with turbulent flow or by means of flow tubes having internals.

Various processes for preparing isocyanates by reacting amines with phosgene in the gas phase are known from the prior art. EP-A-593 334 describes a process for preparing aromatic diisocyanates in the gas phase, wherein the reaction of the diamine with phosgene takes place in a tube reactor which has no moving parts and has a constriction of the walls along the longitudinal axis of the tube reactor. However, the process is problematical since mixing of the feedstreams purely by means of a constriction of the tube wall functions poorly compared to use of an active mixing device. Such poor mixing usually leads to high undesirable solids formation.

EP-A-699 657 describes a process for preparing aromatic diisocyanates in the gas phase, wherein the reaction of the associated diamine with the phosgene takes place in a two-zone reactor in which the first zone, which makes up from 20% to 80% of the total reactor volume, is ideally mixed and the second zone, which makes up from 80% to 20% of the total reactor volume, can be characterized by plug flow. However, because at least 20% of the reaction volume is ideally backmixed, there is a nonuniform residence time distribution which can lead to undesirably increased solids formation.

EP-A-289 840 describes the preparation of diisocyanates by gas-phase phosgenation, in which the preparation takes place, according to the invention, in a turbulent stream at temperatures of from 200° C. to 600° C. in a cylindrical space without moving parts. The omission of moving parts reduces the risk of phosgene escaping. The turbulent flow in the cylindrical space (tube) results, if fluid elements in the vicinity of the wall are disregarded, in a good equalized flow in the tube and thus a narrow residence time distribution which can, as described in EP-A-570 799, lead to a reduction in solids formation.

EP-A-570 799 relates to a process for preparing aromatic diisocyanates in the gas phase, wherein the reaction of the associated diamine with the phosgene is carried out in a tube reactor above the boiling point of the diamine within a mean contact time of from 0.5 to 5 seconds. As described in the document, both reaction times which are too long and reaction times which are too short lead to undesirable solids formation. A process in which the mean deviation of the mean contact time is less than 6% is therefore disclosed. Adherence to this contact time is achieved by carrying out the reaction in a stream in the tube which is characterized either by a Reynolds number of greater than 4000 or a Bodenstein number of greater than 100.

EP-A-749 958 describes a process for preparing triisocyanates by gas-phase phosgenation of (cyclo)aliphatic triamines having three primary amino groups, wherein the triamine and the phosgene are continuously reacted with one another at a flow velocity of at least 3 m/s in a cylindrical reaction space heated to from 200° C. to 600° C.

In the example which is explicitly disclosed, the reaction mixture is passed through a solvent which allows only unspecific separation of the reaction products and leads to a broad quench time distribution.

EP-A-928 785 describes the use of microstructured mixers for the phosgenation of amines in the gas phase. The use of micromixers has the disadvantage that even very small amounts of solids, whose formation cannot be prevented completely in the synthesis of the isocyanate, can lead to blockage of the mixer, which reduces the time for which the phosgenation plant is available.

However, it is in all cases necessary to effectively stop the reaction after an optimal reaction time in order to prevent formation of solids as a result of subsequent reactions of the isocyanate.

EP 1403248 A1 describes the rapid cooling of a reaction mixture comprising isocyanate, phosgene and hydrogen chloride in a cylindrical quench zone. The quench zone comprises at least 2 nozzle heads which in turn comprise one or more individual nozzles. The nozzles are distributed around the external circumference. In the quench zone, the reaction gas is mixed with the sprayed liquid droplets. As a result of vaporization of the liquid, the temperature of the gas mixture is reduced quickly, so that the loss of desired isocyanate product as a consequence of high temperatures is reduced. Furthermore, the nozzle arrangement decreases premature contact of the hot reaction gas with the walls of the quench zone, so that the formation of deposits on the surfaces is reduced.

However, in the embodiment disclosed in the figure, it can be seen that, taking account of the entrainment of the quenching liquid by the inflowing reaction mixture, channels through which the reaction mixture is conveyed without intimate contact with the quenching medium remain open, in particular at the wall of the quench space. This leads to a proportion of unquenched reaction mixture and thus to a broadening of the quench time distribution.

A disadvantage of the process described is the quench times of from 0.2 to 3.0 s, which lead to a significant avoidable loss of isocyanate.

The international patent application WO 2005/123665 describes a process for preparing isocyanates having a constriction between reaction zone and quench. The example which is explicitly disclosed there and has a particular Sauter mean diameter and particular velocity of spraying-in allows quench times of 0.01 second.

However, the measures disclosed there do not enable an optimal quenching effect to be achieved.

It was an object of the invention to develop a process for preparing isocyanates in the gas phase, in which the reaction is stopped within sufficiently short times after the optimal residence time and simple separation of the isocyanate from the other constituents of the reaction mixture can be achieved.

This object has been able to be achieved by carrying out the reaction to a conversion of at least 98% in a reaction zone and stopping the reaction by passing the reaction mixture through a zone into which a liquid is sprayed. This zone will hereinafter be referred to as quench zone. Between the reaction zone and the zone in which the reaction is stopped there is a region which can have a different cross section compared to the quench zone and reaction zone. The cross-sectional area of this region can be smaller or greater than the cross-sectional area of the reaction zone. According to the invention, the gaseous reaction mixture is passed through a curtain of quenching liquid which fills the entire cross-sectional area of the quench zone.

As reaction zone, it is possible to use tube reactors, flow tubes with or without internals or plate reactors.

The reaction of the amine with the phosgene in the gas phase can be carried out under the known conditions.

Mixing of the reaction components amine and phosgene can be effected before or in the reactor. Thus, it is possible for the reactor to be preceded by a mixing unit, for example a nozzle, as a result of which a mixed gas stream comprising phosgene and amine enters the reactor.

In an embodiment of the process of the invention, the phosgene stream is firstly distributed very homogeneously over the entire width of the reactor by means of a distributor element. The amine stream is fed in at the beginning of the reactor where a distributor channel having holes or mixing nozzles is installed in the reaction channel, with this distributor channel preferably extending over the entire width of the reactor. The amine, which may, if appropriate, be mixed with an inert medium, is fed through these holes or mixing nozzles into the phosgene stream.

The inert medium is a medium which is gaseous at the reaction temperature and does not react with the starting materials. For example, it is possible to use nitrogen, noble gases such as helium or argon or aromatics such as chlorobenzene, dichlorobenzene or xylene. Preference is given to using nitrogen as inert medium.

The process of the invention can be carried out using primary amines, preferably diamines or triamines and particularly preferably diamines, which can preferably be converted into the gas phase without decomposition. Particularly suitable amines are amines, in particular diamines, based on aliphatic or cycloaliphatic hydrocarbons having from 1 to 15 carbon atoms. Examples are 1,6-diaminohexane, 1-amino-3,3,5-trimethyl-5-aminomethylcyclohexane (IPDA), 4,4′-diaminodicyclohexylmethane, 1,3- or 1,4-(isocyanatomethyl)cylohexane (BIC) and 3 (or 4), 8 (or 9)-bis(aminomethyl)tricyclo[5.2.1.0^{2.6}]decane isomer mixtures. Preference is given to using 1,6-diaminohexane (HDA).

The process of the invention can also be carried out using aromatic amines which can preferably be converted into the gas phase without decomposition. Examples of preferred aromatic amines are toluenediamine (TDA), preferably 2, 4 or 2,6 isomers or mixtures thereof, diaminobenzene, naphthalenediamine (N DA) and 2,4′- or 4,4′-methylene(diphenylamine) (MDA) or isomer mixtures thereof.

In the process of the invention, it is advantageous to use phosgene in an excess over the amino groups. The molar ratio of phosgene to amino groups is usually from 1.1:1 to 20:1, preferably from 1.2:1 to 5:1.

To carry out the process of the invention, it can be advantageous to preheat the streams of the reactants, usually to temperatures of from 100 to 600° C., preferably from 200 to 500° C., prior to mixing. The reaction in the reaction channel usually takes place at a temperature of from 150 to 600° C., preferably from 250 to 500° C. The process of the invention is preferably carried out continuously.

The reaction of phosgene with amine in the reaction space takes place at absolute pressures of from >0.1 bar to <20 bar, preferably from 0.5 bar to 15 bar and particularly preferably from 0.7 to 10 bar. In the case of the reaction of (cyclo)aliphatic amines, the absolute pressure is very particularly preferably in the range from 0.7 bar to 5 bar, in particular from 0.8 to 3 bar, especially from 1 to 2 bar and very especially from 1.1 to 1.5 bar.

In a preferred embodiment, the dimensions of the reactor and the flow velocities are chosen so that turbulent flow, i.e. flow having a Reynolds number of at least 2300, preferably at least 2700, particularly preferably at least 10 000, with the Reynolds number being formed using the hydraulic diameter of the reactor, prevails. The Reynolds number determines the flow regime and thus the residence time distribution in the reaction tube (H. Schlichting: Grenzschichttheorie, Verlag G. Braun, 1982; M. Baerns: Chemische Reaktionstechnik, Georg Thieme Verlag Stuttgart, 1992). The gaseous reactants preferably travel through the reactor at a flow velocity of from 2 to 220 meters/second, preferably from 20 to 150 meters/second, particularly preferably from 30 to 100 meters/second.

In the process of the invention, the mean contact time is generally from 0.05 to 5 seconds, preferably from 0.06 to 1 second, particularly preferably from 0.1 to 0.45 second. For the purposes of the invention, the mean contact time is the period of time from commencement of mixing of the starting materials to termination of the reaction by the quench. In a preferred embodiment, the flow in the process of the invention is characterized by a Bodenstein number of greater than 10, preferably greater than 100 and particularly preferably greater than 500. The Bodenstein number is a measure of the degree of backmixing in the flow apparatus. The backmixing decreases with increasing Bodenstein number (M. Baerns: Chemische Reaktionstechnik, Georg Thieme Verlag Stuttgart, 1992).

As indicated above, a quench zone is arranged at the end of the reactor which may be a tube reactor operated with turbulent flow, a flow tube having internals or a plate reactor.

The term reaction space refers to the volume in which at least 98% of the conversion, i.e. the consumption of the amine used, takes place, preferably at least 99%, particularly preferably 99.5%, very particularly preferably 99.7%, in particular 99.9% and especially 99.99%.

The invention accordingly provides a process for preparing isocyanates by reacting amines with phosgene in the gas phase in at least one reaction zone, with the reaction mixture being passed through at least one zone into which at least one liquid is sprayed to stop the reaction, in which the reaction mixture is passed through a closed curtain of quenching liquid which completely fills the cross section of the quench zone.

The change in the flow cross section between reaction zone and quench zone is set as a function of the other process engineering parameters and the absolute size of the apparatus. Thus, in the case of small apparatus dimensions and/or isocyanates which have a strong tendency to form deposits, it can be advantageous, for example, to provide a widening of the cross section between reaction zone and quench zone in order to avoid blockage of the cross section. In the case of a widening of the cross section, it should be ensured that the flow is separation-free, because otherwise the formation of deposits likewise has to be expected. The measures necessary for achieving separation-free flow, in particular the required angles at transitions within or between the components, are known per se to those skilled in the art.

On the other hand, in the case of sufficiently large apparatus dimensions or isocyanates which have only a small tendency to form deposits, a constant or preferably narrowing flow cross section between reaction zone and quench zone is preferable.

Isocyanates which have a strong tendency to form deposits are, in particular, monoisocyanates and (cyclo)aliphatic isocyanates, in particular hexamethylene 1,6-diisocyanate.

In contrast, isocyanates which have a low tendency to form deposits are, for example, aromatic isocyanates and in particular tolylene diisocyanate.

As a general rule, the tendency of isocyanates to form deposits increases with increasing functionality, increasing reactivity and/or increasing molecular weight.

A narrowing of the flow cross section is preferably chosen so that the reaction gas on leaving the constriction is, firstly, appreciably cooled and, secondly, has a sufficiently high flow velocity to effect effective secondary atomization of the quenching liquid. For the present purposes, secondary atomization means that liquid droplets produced, for example, by means of atomizer nozzles are broken up further by forces in the gas stream, in particular the aerodynamic forces, so that a greater heat transfer and mass transfer area is obtained.

Both requirements can be achieved by setting the velocities of the stream of reaction mixture according to the boundary conditions of the cross sections:

In the case of a widening of the flow cross section in the direction of flow of the reaction mixture, the Mach number of the stream of reaction mixture at the inlet into the quench zone is generally from 0.05 to <1.0, preferably from 0.1 to <1.0, particularly preferably from 0.2 to <1.0 and very particularly preferably from 0.3 to <1.0.

In the case of a narrowing of the flow cross section in the direction of flow of the reaction mixture, the Mach number downstream of the constriction in the cross section can additionally be at least 1.0, for example up to 5.0, preferably up to 3.5, particularly preferably up to 2.5 and very particularly preferably up to 1.5. Adiabatic after-expansion of the reaction mixture after leaving the reaction zone and before meeting the quenching liquid is conceivable. This has the consequence that the precooled reaction mixture is subject to a compression pulse shortly before meeting the quenching medium and the temperature increase caused by this is taken up by the quenching process.

The Mach number is the ratio of the local flow velocity to the local speed of sound in the reaction mixture. The Mach number requirements directly determine, on the basis of the mass balance of the given stream, pressure and temperature, the size of the inlet cross section into the quench zone.

The ratio of the narrowest flow cross sections in the reaction zone and the quench zone is, in the case of sufficiently large apparatus dimensions or isocyanates which display only a low tendency to form deposits, from 1/1 to 10/1, preferably from 1.2/1 to 10/1, particularly preferably from 2/1 to 10/1 and very particularly preferably from 3/1 to 10/1. In the case of small apparatus dimensions which are susceptible to blockage or isocyanates which have a strong tendency to form deposits, a widening of the flow cross section between reaction zone and quench zone of from 1/1 to 1/10, preferably from 1/1.2 to 1/10, particularly preferably from 1/2 to 1/10 and particularly preferably from 1/3 to 1/10, based on the flow cross-sectional area of the reaction tube, is advantageous.

For the purposes of the present invention, dimensions susceptible to blockage are the smallest diameters or slit dimensions in each case in which deposits can be formed.

The transition between reaction zone and quench zone is preferably configured in the form of a cone. However, tapered shapes having an oval or ellipsoidal cross section or concave or convex transitions, i.e., for example, hemispherical spaces, are also conceivable.

In the quench zone, the reaction mixture which consists essentially of the isocyanates, phosgene and hydrogen chloride is intensively mixed with the liquid sprayed in.

According to the invention, the mixing of reaction mixture and liquid has to occur so that the reaction mixture cannot partly bypass the quenching liquid. This ensures that the entire reaction mixture is cooled within a very short time. Furthermore, it is ensured that this cooling occurs uniformly, i.e. with a small deviation from the mean cooling time.

This has not been able to be ensured by the prior art, since the nozzles disclosed in the prior art do not ensure that no channels through which the reaction mixture can flow past the quenching medium remain open or that the time between entry into the quench zone and contact with the quenching medium is sufficiently short and very uniform.

Mixing is carried out so that the temperature of the reaction mixture is reduced from an initial 150 to 600° C., preferably 250 to 500° C., by 50-300° C., preferably by 100 to 250° C., down to 100-200° C., preferably 140-180° C., and part or all of the isocyanate comprised in the reaction mixture goes over into the sprayed-in liquid droplets as a result of condensation while the phosgene and the hydrogen chloride remain essentially completely in the gas phase.

The proportion of the isocyanate comprised in the gaseous reaction mixture which goes over into the liquid phase in the quench zone is preferably from 20 to 100% by weight, particularly preferably from 50 to 99.5% by weight and in particular from 70 to 99% by weight, based on the isocyanate comprised in the reaction mixture.

The proportion of the hydrogen chloride comprised in the gaseous reaction mixture which goes over into the liquid phase in the quench zone is preferably less than 20% by weight, particularly preferably less than 15% by weight, very particularly preferably less than 10% by weight and in particular less than 5% by weight.

The proportion of the phosgene comprised in the gaseous reaction mixture which goes over into the liquid phase in the quench zone is preferably less than 20% by weight, particularly preferably less than 15% by weight, very particularly preferably less than 10% by weight and in particular less than 5% by weight.

The reaction mixture preferably flows through the quench zone from the top downward. At the outlet from the quench zone, there is a collection vessel in which the liquid phase is precipitated, collected and removed via an outlet and is subsequently worked up. The remaining gas phase is removed via a second outlet and is likewise worked up.

The liquid droplets of the quenching medium are produced by means of suitable nozzles, for example single- or two-fluid atomizer nozzles, preferably single-fluid atomizer nozzles, and preferably have a Sauter mean diameter D₃₂ of from 5 to 5000 μm, particularly preferably from 5 to 500 μm and in particular from 5 to 250 μm.

The Sauter mean diameter D₃₂ (SMD) describes, except for a constant factor, the ratio of the mean droplet volume to the mean droplet surface area (cf. K. Schwister: Taschenbuch der Verfahrenstechnik, Fachbuchverlag Leipzig, Carl Hanser Verlag 2003) and is thus the important parameter of the droplet size distribution produced in the quenching process. It is the droplet diameter at which the volume/surface area ratio is the same as that for the sum of all droplets in the ensemble under consideration and indicates the degree of fineness of the atomization with regard to the reaction surface area.

The width of the droplet size distribution should be very low because droplets which are too large cannot bring about a rapid temperature decrease and droplets which are too small can subsequently be separated from the gas stream only with increased difficulty.

The atomizer nozzles produce, depending on the embodiment, a spray cone angle of from 10 to 140°, preferably from 10 to 120°, particularly preferably from 10° to 100°. FIG. 7 shows the definition of the spray cone angle α (alpha).

The spray image is, for the present purposes, the part of an area perpendicular to the spray axis (in the case of rotationally symmetric nozzles) or perpendicular to the mirror plane (in the case of mirror-symmetric nozzles) through which the liquid droplets pass. The outer contour of the spray image is generally circular (in the case of solid cone nozzles) or annular (in the case of hollow cone nozzles). However, it can also be oval or elliptical to rectangular (e.g. in the case of flat jet nozzles).

The envelope of the sprayed droplets is generally conical and in the vicinity of the nozzle ideally forms a cone. A hollow cone is also conceivable. However, depending on the shape of the quench zone, it can also be advantageous to use spray nozzles which produce a nonconical envelope. Furthermore, fan-shaped envelopes, for example as produced by slit nozzles or flat jet nozzles, are conceivable.

To set the necessary droplet size, single-fluid atomizer nozzles are generally operated at an overpressure relative to the quench zone pressure of at least 1 bar, preferably at least 4 bar, particularly preferably at least 10 bar, very particularly preferably at least 20 bar and in particular at least 50 bar.

In the case of single-fluid atomizer nozzles, it is generally sufficient to employ an overpressure of not more than 1000 bar, preferably not more than 500 bar, particularly preferably not more than 200 bar, very particularly preferably not more than 100 bar and in particular not more than 80 bar.

In the case of two-fluid atomizer nozzles, the nozzle can, on the liquid side, be operated either as a pressure nozzle or as a suction nozzle, i.e. the admission pressure of the liquid relative to the quench zone pressure can be positive or negative. The atomizer gas generally has an admission pressure which is sufficiently high for the ratio of admission pressure to quench zone pressure to be greater than the critical pressure ratio, preferably greater than twice the critical pressure ratio and particularly preferably greater than four times the critical pressure ratio. The critical pressure ratio indicates the pressure ratio at and above which the pressure in the narrowest cross section of the atomization gas channel is independent of the pressure downstream of the nozzle.

The velocity at which the droplets leave the nozzle depends on the type of atomization and is generally at least 15 m/s, preferably at least 40 m/s and particularly preferably at least 100 m/s. The upper limit of the velocity is not critical. A velocity of up to 350 m/s is frequently sufficient.

Between the reaction zone and the quench zone, there can preferably be a constriction in the cross section through which depressurization, associated with a decrease in concentration of the reactants, and a first decrease in temperature of the reaction gas, is achieved. Furthermore, the reaction gas stream leaving the constriction in the cross section with an increased velocity effects additional secondary atomization of the quenching liquid on meeting the quenching liquid spray.

The large specific surface area of the liquid droplets and the high relative velocities between reaction gas and quenching liquid intensify the mass transfer and heat transfer between reaction gas and quenching liquid. As a result, not only are bypass flows of the reaction mixture avoided but the contact times necessary for cooling of the reaction mixture are greatly reduced and the loss of desired isocyanate product due to further reaction to form by-products is minimized.

The velocity of the reaction gas stream in the narrowest cross section is preferably more than 20 m/s, particularly preferably more than 50 m/s, in particular more than 100 m/s, and an upper limit on it is imposed by the speed of sound in the reaction gas mixture under the respective conditions. In the case of critical flow through the narrowest cross section, after-expansion and further acceleration of the reaction gas mixture occur downstream of the narrowest cross section.

The free flow cross section in the quench zone is, based on the free flow cross section in the reaction zone, generally from 25/1 to 1/2, preferably from 10/1 to 1/1.

The arrangement of the atomization nozzles in the quench zone is selected so that bypass flow of the reaction mixture past the quenching liquid is largely avoided. This is achieved by the quenching liquid droplets in the quench zone forming a closed curtain which separates the region of one or more reaction mixture inlets into the quench zone completely from the region of the outlets from the quench zone. As a result, the entire reaction mixture has to penetrate through the curtain formed by the quenching liquid, i.e. the totality of the time average volumes through which droplets from the quench nozzles pass, and is thus cooled efficiently.

The liquid curtain can have different shapes depending on the atomization devices used. Thus, for example, atomization devices having a circular spray image (for example a conical envelope) or else an elliptical spray image can be used. In addition, it is also possible to use slit-shaped nozzles having an approximately oval or elliptical to rectangular spray image (fan-shaped envelope). In the case of a conical or elliptical conical envelope, the cone can be a hollow cone or a solid cone.

The atomizer nozzles are arranged in the quench zone so that the isosurfaces of the quenching liquid volume fraction which define the envelope of the individual nozzles together with the quench zone wall and the reaction gas inlet envelop a closed volume. The spraying-in direction of the atomizer nozzles, which in the case of conical nozzles is defined by the central axis of the spray cone, and the main flow direction of the gas in the quench zone can form an angle of from 0° to 180°, preferably from 0° to 90°, particularly preferably from 0° to 60°. Here, an angle of 0° means that the atomizer nozzle axis is exactly parallel to the main flow direction and the nozzle sprays in the direction of the main flow, while an angle of 90° means that the atomizer nozzle axis is exactly perpendicular to the main flow direction in the quench zone. An angle of 180° means that the atomizer nozzle sprays the quenching liquid in a direction exactly opposite to the main flow direction.

The curtain of quenching liquid can be produced by means of one or more devices for atomizing the quenching liquid. The ratio of the number of atomization devices to the number of reaction mixture inlets into the quench zone is from 10/1 to 1/10, preferably from 4/1 to 1/4, particularly preferably from 4/1 to 1/1, very particularly preferably from 3/1 to 1/1 and in particular from 2/1 to 1/1.

In a preferred embodiment (FIG. 1) having one nozzle, the quench nozzle 2 is located coaxially in the middle of a cylindrical or conical quench zone 5. FIG. 1 depicts a quench zone made up of a cylinder with a superposed cone. The reaction mixture 3 is introduced via an annular gap 4 coaxially to the quench nozzle 2 into the quench zone 5. The quench zone wall 7 and the spray cone 6 form a narrowing space 8 into which the reaction mixture flows. As a result of these structural measures, the reaction mixture then has to flow through the curtain formed by the spray cone. In this preferred embodiment, the spray cone angle has to be greater than the cone angle of the quench zone wall.

In a second preferred embodiment (FIG. 2) having one nozzle, the nozzle 2 is likewise located coaxially in the middle of a cylindrical or conical quench zone 5. Here, the reaction mixture is introduced via an inlet 3 into the quench zone at an angle β (beta) to the spray nozzle axis, with the angle β being from 0° to 90°, preferably from 45° to 90°, particularly preferably from 70° to 90°. An angle β of 0° here means parallel to the spray nozzle axis and an angle β of 90° means perpendicular to the spray nozzle axis. In a particularly preferred arrangement, the reaction mixture stream enters the quench zone tangentially. This means that the reaction mixture stream is not directed straight at the spray nozzle axis but instead its direction forms an angle of from 5° to 45°, preferably from 10° to 45°, particularly preferably from 20° to 45° and very particularly preferably from 30° to 45°, with the connecting axis of the reaction mixture inlet 3 with the spray nozzle axis. The reaction mixture then again flows through the narrowing space 8 formed by the spray cone 6 and the quench zone wall 7 and finally penetrates through the quenching liquid curtain. In this preferred embodiment, the spray cone angle has to be greater than the cone angle of the quench zone wall.

In a further preferred arrangement having a plurality of atomization devices 2, from 2 to 10, for example, atomization nozzles 2 are arranged on a ring around the inlet for the reaction mixture 3 (FIGS. 3a and 3b). In FIG. 3a, six atomization nozzles are shown by way of example. The spray nozzles produce, due to superposition of the individual spray images, an elliptical or circular spray image 6. The reaction mixture inlet 3 is located in the interior of the ring. The axis of the spray cone is inclined at an angle γ (gamma) to the entry direction of the reaction mixture. Gamma γ is from 0°, so that the quenching liquid is sprayed in parallel to the reaction mixture, to 90°, so that the quenching liquid is sprayed in perpendicular to the reaction mixture, preferably from 0° to 60°, particularly preferably from 0° to 45°. The advantage of a plurality of nozzles is that smaller nozzles which generally produce smaller droplets and thus make more rapid quenching of the liquid possible can be used. Once again, a suitable combination of the quench zone shape and arrangement of the atomization devices ensures that a closed spray curtain is formed.

FIG. 4 shows a variant of the arrangement of FIG. 3 having a constriction in the cross section 11 between reaction zone and quench zone.

This constriction in the cross section leads to acceleration of the reaction mixture and thereby to a decrease in pressure, which effects cooling of the reaction mixture. As a result of the acceleration, the reaction mixture can reach a velocity of up to Mach 1.0 in the narrowest cross section. Downstream of the narrowest cross section, velocities of greater than Mach 1.0 can also be obtained.

As a result of this cooling, the reaction mixture is subject to lower thermal stress up to the quenching process. In addition, the increased velocity of the reaction mixture effects secondary atomization of the quenching droplets and thus improves heat and mass transfer between reaction gas mixture and quenching liquid. Although the impingement of reaction mixture and quenching droplets onto one another briefly leads to a temperature increase, this is taken up by the quenching liquid in the quenching process and thus leads to no further thermal stressing of the reaction mixture.

In a further, preferred arrangement, the reaction gas mixture enters the quench zone via a slit at the end face. The slit can be circular or elliptical or form any other curve. The slit width can be variable, but is preferably constant. On both sides of the slit there are, depending on the circumference of the slit, one or more atomizer nozzles which spray quenching liquid in parallel or at an angle γ to the main flow direction of the reaction gas mixture. The angle γ is from 0° to 90°, preferably from 0° to 60°, particularly preferably from 0° to 30°. The spray nozzles on both sides of the slit result in a narrowing flow channel for the reaction gas mixture which is closed off by the meeting of the spray images of the atomizer nozzles. The result is once again a closed curtain through which the reaction mixture has to pass and is thus cooled rapidly. The slit is preferably an annular slit through which the reaction mixture is conveyed and in which at least one spray nozzle for the quenching liquid is located on the inside and, depending on the circumference of the annular slit, a plurality of spray nozzles, for example from 2 to 10, preferably from 2 to 8 and particularly preferably from 3 to 6 nozzles, for the quenching liquid are located on the outside.

In a further preferred embodiment having a plurality of reaction gas inlets 3 and a plurality of atomization devices 2, a plurality of atomization nozzles 2 and reaction gas inlets 3 are located on the end face 10 of the quench zone. The atomization devices 2 and the reaction mixture inlets 3 are preferably distributed uniformly (FIG. 5). The atomization devices once again form a closed curtain similar to that in FIG. 3a. Preference is here given to an arrangement of the atomization devices 2, as shown in FIG. 5, in which the atomization devices form an outer ring, i.e. are located between the side wall of the quench zone 7 and the reaction mixture inlets 3, so that it is ensured that the reaction mixture does not come into contact with the wall but impinges on the quenching medium.

A further preferred embodiment is shown in FIG. 6. Here, the reaction gas 3 is conveyed along the longitudinal axis of the quench zone in which a curtain made up of a plurality of, in FIG. 6 four, overlapping fan-shaped envelopes is present perpendicular to the flow direction of the reaction gas. These overlapping fan-shaped envelopes fill out the entire cross section of the quench nozzle so that the reaction gas comes into contact with the quenching liquid.

The spray nozzle axes of the quench nozzles which in FIG. 6 are, for example, installed laterally on the quench zone can particularly preferably enclose an angle with the longitudinal axis of the quench zone of 90°, i.e. be perpendicular to the longitudinal axis of the quench zone. However, it is possible for the spray nozzle axes to enclose an angle of from about −45° to +135° with the longitudinal axis, i.e. be directed opposite to or preferably in the same direction as the flow direction of the reaction gas.

Preference is given to introducing the output from one reaction zone into the quench zone, but it is also possible to feed the outputs from a plurality of reaction zones via one or more inlets into one quench zone.

It is also possible to divide the output from a reaction zone and feed it via a plurality of inlets into one or more quench zones.

The liquid which is sprayed in via the atomizer nozzles has to have a good solvent capability for isocyanates and a low solvent capability for hydrogen chloride and/or phosgene. Preference is given to using organic solvents. In particular, use is made of aromatic solvents which may be substituted by halogen atoms. Examples of such liquids are toluene, benzene, nitrobenzene, anisole, chlorobenzene, dichlorobenzene (ortho, para), trichlorobenzene, xylene, hexane, diethyl isophthalate (DEIP) and also tetrahydrofuran (THF), dimethylformamide (DMF) and mixtures thereof.

In a particular embodiment of the process of the invention, the liquid sprayed in is a mixture of isocyanates, a mixture of isocyanates and solvent or one isocyanate (with the quenching liquid used in each case being able to comprise proportions of low boilers such as HCl and/or phosgene of up to 20% by weight, preferably up to 10% by weight, particularly preferably up to 5% by weight and very particularly preferably up to 2% by weight). Preference is given to using the isocyanate which is prepared in the respective process. Since the reaction is stopped by the reduction in temperature in the quench zone, secondary reactions with the isocyanates sprayed in can be reduced if not ruled out. The advantage of this embodiment is, in particular, that it is not necessary to separate off the solvent.

The temperature of the liquid sprayed in is preferably from 0 to 300° C., particularly preferably from 50 to 250° C. and in particular from 70 to 200° C., so that the desired cooling and condensation of the isocyanate is achieved with the amount of liquid sprayed in. This largely stops the reaction.

The velocity of the reaction gas in the quench zone is preferably greater than 1 m/s, particularly preferably greater than 10 m/s and in particular greater than 20 m/s.

The velocity of the reaction gas in the quench zone is preferably greater than 1 m/s, particularly preferably greater than 10 m/s and in particular greater than 20 m/s. In the case of a constriction in the cross section between reaction zone and quench zone, a velocity up to the speed of sound in the respective system can be reached in the narrowest cross section. A further expansion of the stream between the narrowest cross section and the quench zone can then result in flow velocities above the speed of sound, which cause significant cooling of the gas. In this case, a compression pulse then occurs in the region of the quench zone and this leads to sudden braking of and a pressure increase in the gas.

To achieve rapid cooling of the gaseous reaction mixture in the quench zone and rapid transfer of the isocyanate into the liquid phase, the droplets of the liquid sprayed in have to be finely distributed very quickly over the entire flow cross section of the reaction gas. The desired temperature decrease and the desired transfer of the isocyanate into the droplets is preferably effected in up to 10 seconds, particularly preferably in up to 1 second and in particular in up to 0.2 second. The numerical values given are mean quench times. As a result of the particular configuration of the quench zone, the deviations of the minimum and maximum quench time from this mean are kept small. The standard deviation based on the mean. The relative standard deviation based on the mean of the quench time distribution is not more than 1, preferably not more than 0.5, particularly preferably not more than 0.25 and in particular 0.1. The above times (quench times) are defined as the period of time from when the reaction gas enters the quench region to the point in time at which the reaction gas has experienced 90% of the temperature change from the entry temperature into the quench region to the adiabatic final temperature. The adiabatic final temperature is the temperature which is established when the reaction mixture and the quenching liquid are mixed at the respective flows and entry temperatures under adiabatic conditions and reach thermodynamic equilibrium. The selected periods of time enable loss of isocyanate due to secondary and further reactions to be virtually completely avoided.

The mass ratio of the amount of liquid sprayed in to the amount of the gaseous reaction mixture is preferably from 100:1 to 1:10, particularly preferably from 50:1 to 1:5 and in particular from 10:1 to 1:2.

The liquid phase and gas phase taken from the quench zone are worked up. When a solvent is used as atomized liquid, a separation of isocyanate and solvent is carried out, usually by means of distillation. The gas phase, which comprises essentially phosgene, hydrogen chloride and possibly isocyanate which has not been separated off, can likewise be separated into its constituents, preferably by distillation or adsorption, with the phosgene being able to be recirculated to the reaction and the hydrogen chloride being able to be either utilized for further chemical reactions, processed further to give hydrochloric acid or dissociated into chlorine and hydrogen again.

FIGS. 1 to 5 show embodiments of the process of the invention.

The invention is illustrated by the following examples.

### EXAMPLE 1

In a tube reactor having a diameter of 8 mm and provided with an upstream mixing device, 20 kg/h of reaction gas comprising tolylene diisocyanates, phosgene and hydrogen chloride were produced.

The reaction gas was then fed via an annular slit having an internal diameter (D_{O,I}) of 17 mm and an external diameter (D₁) of 19 mm into the quench zone. In the quench zone there was a single-fluid nozzle which was arranged coaxially in the interior of the annular slit (FIG. 1). The spray cone opening angle of the nozzle was 70°. The nozzle produced droplets having a Sauter mean diameter of about 60 μm. The quench zone comprised a 10 mm (L₁) long cylindrical part having a diameter (D₁) of 19 mm, a subsequent 40 mm long (L₂-L₁) conical part which widened from 19 mm to 70 mm, followed by a 70 mm long (L₃) cylindrical part having a diameter (D₂) of 70 mm and finally a further conical part having an angle of taper of 60° and a final diameter of 12 mm (not shown in FIG. 1). The amount of liquid sprayed in was 17.4 kg/h. The quenching liquid sprayed in was monochlorobenzene. The temperature of the reaction gas on entry into the quench zone was 363° C. and the pressure of the gas was 1.35 bar. The entry temperature of the quenching liquid was 100° C., and the exit velocity of the liquid droplets from the spraying nozzle was about 60 m/s. The residence time of the reaction gas in the front conical region of the quench zone was about 0.029 s. Here, the temperature of the quench gas dropped to about 156° C. The desired temperature decrease occurred in about 8 ms. The amount of tolylene diisocyanate in the reaction gas mixture decreased by 80% compared to the concentration on entering the quench zone.

### LIST OF FIGURES

FIG. 1: Quench nozzle coaxially over quench zone, introduction of the reaction mixture via annular slit

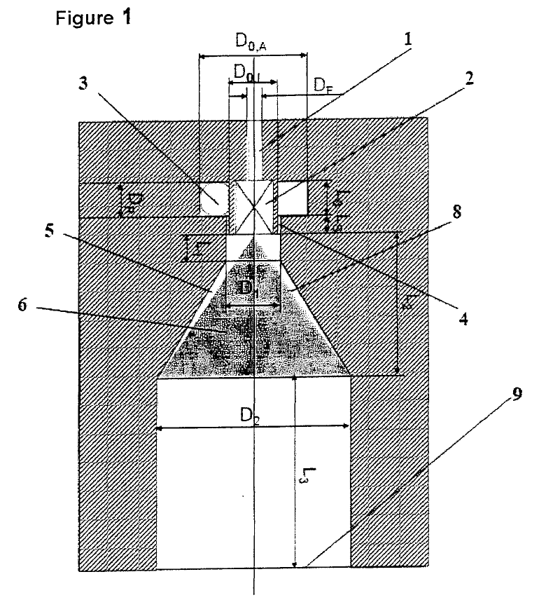

FIG. 2: Quench nozzle coaxially over quench zone, introduction of the reaction mixture at angle β (beta)

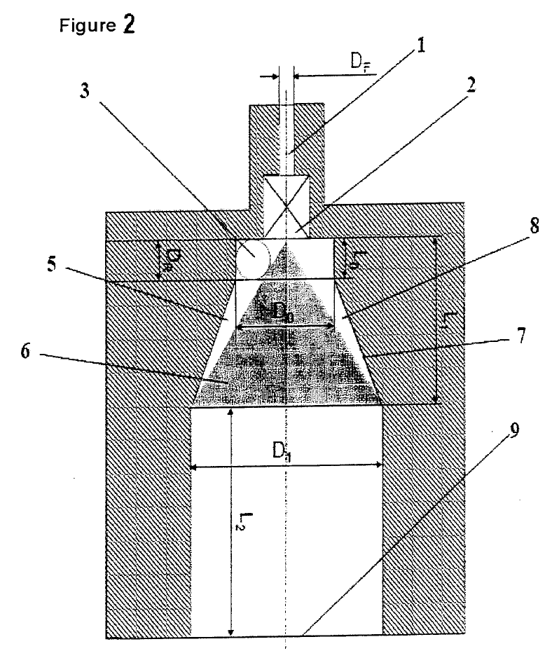

FIG. 3a: Introduction using a plurality of atomization nozzles

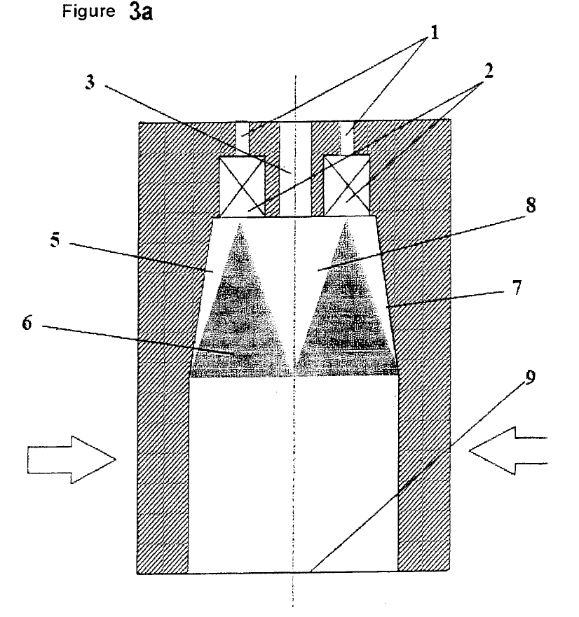

FIG. 3b: Section 1-1 in FIG. 3a

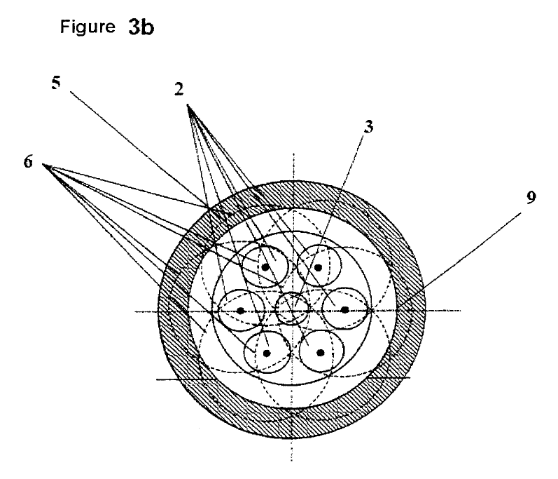

FIG. 4: Constriction in the cross section between reaction zone and quench zone

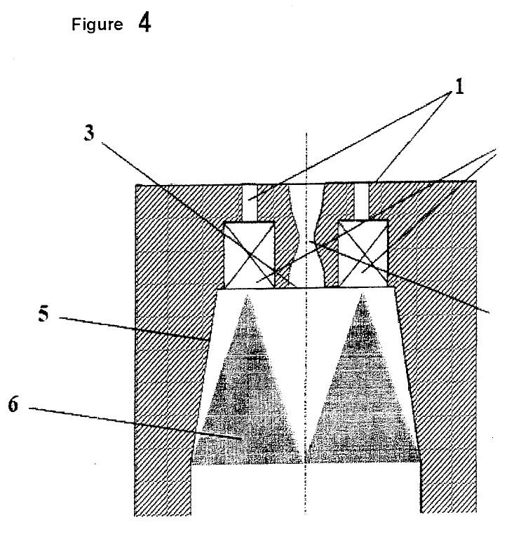

FIG. 5: Introduction using a plurality of reaction mixture inlets and atomization nozzles

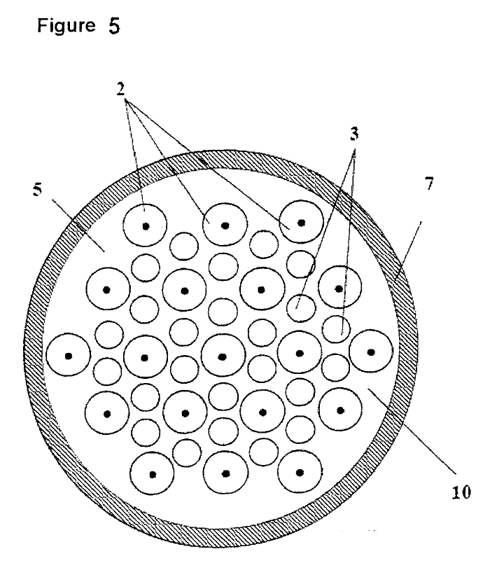

FIG. 6: Introduction of the quenching medium perpendicular to the flow direction of the reaction gas. At left: side view, at right: view perpendicular to the section A-A

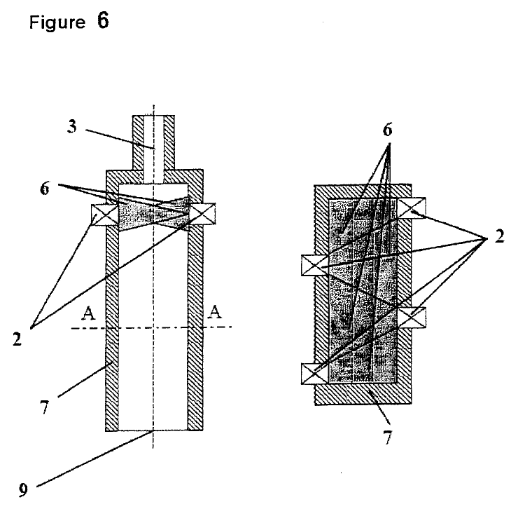

FIG. 7: Definition of the spray cone angle α (alpha)

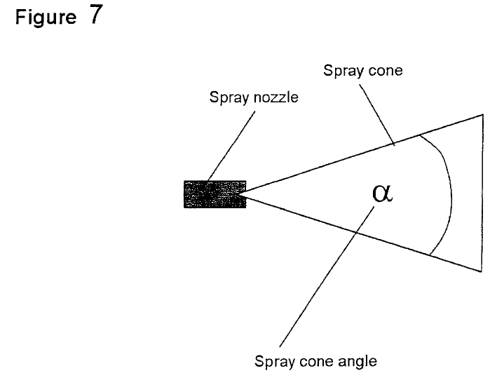

### LIST OF THE REFERENCE NUMERALS IN THE FIGURES

1 Quenching liquid inlet
2 Atomization device
3 Reaction mixture inlet
4 Annular slit
5 Quench zone
6 Spray cone
7 Wall
8 Enclosed space
9 Liquid and gas outlet
10 End face of the quench zone
11 Constriction in the cross section

1. A process for preparing isocyanates comprising:
reacting amines with phosgene in the gas phase in at least one reaction zone, with the reaction mixture being passed through at least one zone into which at least one liquid is sprayed to stop the reaction, wherein the reaction mixture is passed through a closed curtain of quenching liquid which completely fills the cross section of the quench zone.

2. A process for preparing isocyanates, comprising
reacting amines with phosgene in the gas phase in at least one reaction zone, with the reaction mixture being passed through at least one quench zone into which at least one quenching liquid is sprayed to stop the reaction, wherein the quench zone has a cylindrical or conical shape and the quenching liquid is sprayed therein in such a way that the spray image of the quenching liquid forms a closed space with the wall of the quench zone and the reaction mixture is fed into this space.

3. The process according to claim 2, wherein the quenching liquid is sprayed in co-axially by means of a spray device.

4. The process according to claim 2, wherein the reaction mixture is introduced into the quench zone at an angle β (beta) ranging from 45° to 90° relative to the spray nozzle axis of said spray device.

5. The process according to claim 2, wherein the reaction mixture is introduced tangentially into the quench zone.

6. The process according to claim 1, wherein quenching of the reaction mixture occurs within from 0.001 to 0.2 seconds.

7. The process according to claim 6, wherein the relative standard deviation of the quench time is less than 1.

8. The process according to claim 1, wherein the stream of reaction mixture at the inlet into the quench zone has a velocity ranging from Mach 0.05 to Mach 1.0.

9. The process according to claim 1, wherein the stream of reaction mixture at the inlet into the quench zone has a velocity ranging from at least Mach 1.0 to Mach 5.0.

10. The process according to claim 1, wherein the ratio of flow cross section of the narrowest flow cross section between reaction zone and quench zone ranges from 10/1 to 1/10.

11. The process according to claim 1, wherein the ratio of flow cross section in the quench zone to the free flow cross section in the reaction zone ranges from 25/1 to 1/2.

12. The process according to claim 1, wherein the reaction mixture has a temperature ranging from 150 to 600° C. when it enters the quench zone.

13. The process according to claim 1, wherein the quenching medium is comprised of liquid droplets having a Sauter mean diameter D32 ranging from 5 to 5000 μm.

14. The process according to claim 4, wherein the liquid droplets of the quenching medium leave the nozzle at a velocity of at least 15 m/s.

15. The process according to claim 1, wherein the quench zone is provided with a plurality of atomization devices and a plurality of mixture inlets such that the ratio of the number of atomization devices to the number of reaction mixture inlets into the quench zone ranges from 10/1 to 1/10.
